(19) United States
(12) Patent Application Publication
(10) Pub. No.: US 2009/0062781 A1
(43) Pub. Date: Mar. 5, 2009

(54) METHOD OF USING GEL SHEETS FOR LASER TREATMENT

(75) Inventors: Wolfgang Strasser, Freiburg (DE); Heinrich Wokalek, Freiburg (DE)

Correspondence Address: ROTHWELL, FIGG, ERNST & MANBECK, P.C., 1425 K STREET, N.W., SUITE 800, WASHINGTON, DC, 20005, US

(73) Assignee: ED. GEISTLICH SOEHNE AG FUR CHEMISCHE INDUSTRIE, Wolhusen (CH)

(21) Appl. No.: 12/268,135

(22) Filed: Nov. 10, 2008

Related U.S. Application Data
(62) Division of application No. 10/442,262, filed on May 21, 2003.
(60) Provisional application No. 60/382,373, filed on May 23, 2002.

(51) Int. Cl. A61B 18/20

(52) U.S. Cl. 606/9

(57) ABSTRACT

A method and combination for performing laser therapy, involving the steps of providing a sheet of clear film of appropriate size and thickness, and placing the sheet of clear film over an area of a skin to be treated prior to laser therapy. The method may further include steps of replacing the sheet with a new sheet of clear film, periodically during the therapy.

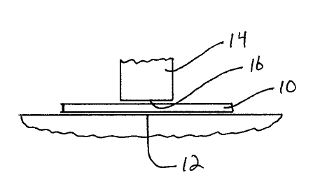

### CROSS-REFERENCE TO RELATED APPLICATIONS

This application is a divisional of U.S. application Ser. No. 10/442,262, filed May 21, 2003, and claims priority from United States provisional application entitled, “Use of Gel Sheets in Laser Treatment”, Ser. No. 60/382,373, filed May 23, 2002, the contents of which are hereby incorporated by reference.

### BACKGROUND OF THE INVENTION

1. Field of the Invention

The present invention generally relates to laser treatment of a patient.

2. Description of the Related Art

Lasers have been used in medical application for decades. Lasers are particularly usefully in dermatological applications because of their ability to reduce scar tissue and improve the cosmetic appearance of the skin. For instance, photothermolysis lasers (PTL) devices can provide up-to-date treatment available for dermatological applications such as the removal of vascular lesions, some benign pigmented lesions, tattoos and also for hair removal. However, a number of problems are associated with the use of PTL devices.

PTL devices work at high temperature, which naturally leads to unpleasant or painful sensations for patients receiving treatment. As a result, compliance and the success rate of treatment are reduced, and the time involved in treatment is increased.

To attempt to solve this problem, cooling fans, ice packs, or refrigeration units have used before applying the laser. However, the application of such cooling processes make treatment considerably more complicated. More recently, “shots” of liquid nitrogen have been directed at the skin shortly before the laser therapy is applied. These techniques are deficient because they are work-intensive and time-consuming, disrupt practice routine terribly, and are ultimately unpleasant, or even very painful, for the patients (e.g., liquid nitrogen application).

Furthermore, it has been found that PTL treatment on previously cooled skin is less effective. A skin surface that has been deeply cooled before laser application reacts more slowly and less favorably to photothermolysis than a surface that has not been so deeply cooled. Thus, the energy densities often have to be increased to achieve the desired effect with the laser, which, in turn increases the pain the patient feels.

Effective PTL therapy requires transparency of the skin, which is a second problem. PTL utilizes different wavelengths of visible light, and in order to achieve the best effect in the target area of the skin (i.e., deep epidermis and corium), the skin surface must be as transparent as possible to permit the laser beam to penetrate down to the tissue to be treated. The higher the transparency, the lower the energy density needed for successful treatment, and the lower the damage that is caused to the surface epithelium, which should not be harmed. Only in these circumstances is laser therapy less traumatic and more selectively effective than the surgical alternative. The important structures in this respect are the vessels, pigment deposits in the epidermis and the superficial corium, hair follicles, and exogenous pigment (tattoos).

A third problem with PTL devices is that many PTL devices require continuous and direct contact with skin surface in order to achieve successful laser therapy and without burning the skin. Abundant amounts of a semi-liquid ultrasound gel are usually applied to the skin to allow continuous and direct contact with the laser. Besides the unaesthetic appearance and the unpleasant sensation for the patient, in this case, laser treatment can literally degenerate into a slime bath. Also, therapists must take the greatest care to ensure sufficient gel remains under the laser head to maintain the contact technique. The constant movements of the applicator alone are sufficient to push the gel to one side so it has to be scooped up and put back under the applicator. This process is tedious, time-consuming, unprofessional and blocks the view of the area being treated. Such semi-liquid gels are considered a particular hindrance, not only to techniques for treating vascular changes, such as reticular varices and couperose, but also for pigmentation treatments, such as treating lentigenes and epilation for hirsutism. When semi-liquid gels are used, a smeary layer develops on the skin, which also detracts from the transparency and visibility during laser therapy.

Thus, there is a need for new and improved methods for performing PTL therapy, which are more efficient and less painful or uncomfortable for the patient.

### BRIEF DESCRIPTION OF THE DRAWING

The FIGURE schematically illustrates one embodiment of the invention.

### SUMMARY OF THE INVENTION

In accordance with the present invention, a method for performing laser therapy includes providing a sheet of solid or gel clear film material, which is applied over an area of a service of a patient to be treated, prior to performing laser treatment to said area.

### DETAILED DESCRIPTION OF THE PREFERRED EMBODIMENTS

The present method is for performing laser therapy to an area of a surface of a patient to be treated. The method involves providing a sheet of clear film material, wherein the sheet of clear film material is a solid or gel. The sheet of clear film material 10 is applied over an area of a surface 12 of a patient to be treated. The surface can be any area of a patient to be treated, such as a skin surface of the patient or an eye surface of patient. In preferred embodiments, the method involves treatment of a skin surface of a patient.

In accordance with an embodiment of the present invention, a skin surface area to be treated with a laser is covered with a clear film of hydrophilic, organic transparent gel, which may be in the form of a sheet or strip. Preferably, the gel material is a transparent, clear film, having a thickness within the range of about 0.1-25 mm, preferably about 0.5-10 mm, and more preferably, within a range of about 1-5 mm.

According to an embodiment of the present invention, the gel comprises a mixture of a hydrophilic polymer and at least one gellable substance of high molecular weight. Particularly preferred gel compositions and films are disclosed in U.S. Pat. Nos. 4,556,056, 4,905,705 and 5,076,265, the entire contents of each of which are incorporated herein by reference. As an example, a commercially available polyacrylimide agar gel film called GELIPERM® is a suitable material. GELIPERM® is a trademark of and manufactured by Ed Geistlich Soehne AG fuer Chemische Industrie, Wolhusen, Switzerland, the assignee of the present invention.

According to an embodiment of the invention, prior to treatment, a clear film is applied to a surface of skin to be subjected to laser treatment with a PTL device. Laser therapy is applied to the skin through the clear gel film.

According to an embodiment of the present invention, provided is a method for performing laser therapy, comprising the steps of providing a clear film of appropriate size and thickness, and placing said film over an area of a skin to be treated prior to laser therapy. The method may further include steps of replacing the film with a new clear film, periodically through the therapy.

Further applications and advantages of the present invention are discussed below.

According to an embodiment of the present invention, a clear film is applied to the skin prior to therapy so that the laser may be kept in constant contact with the area of the patient's skin that to be treated. Laser 14 used for therapy may be commercially available PTL devices, such as an argon laser, Q-switched ruby laser, diode laser (Medio-Star, epilation and Dornier; starburst veins) and Aramis wrinkle laser. Acceptable films are described in gel compositions and films are disclosed in U.S. Pat. Nos. 4,556,056, 4,905,705 and 5,076,265, which are already incorporated herein by reference. However, one having ordinary skill in the art will understand that other films may be substituted having similar qualities and characteristics, as described herein.

The film sheet may be cut or shaped to an appropriate size for covering the region of the skin to be treated, or may be pre-cut or pre-sized. The gel material should be a transparent, clear film, having a thickness within the range of about 0.1-25 mm, preferably about 0.5-10 mm, and more preferably, within a range of about 1-5 mm. The film may be disposable. Transparency should be optimized.

A commercially available product called Geliperm® has an extremely high transparency characteristic, and is therefore, an exemplary preferred film. Contact between the laser applicator (e.g., PTL device) and the skin surface is easily maintained over any parts of the body, e.g. also over the nose and the chin. Thus, treatment with PTL that may require cooling, transparency, or skin contact for successful application, can be applied problem-free to all parts of the body.

The application of the film prior to treatment allows for pain-free and long-lasting cooling of the skin. One novel result of the present invention is the reduction of the smell caused by heating structures containing keratin. Furthermore, heat erythema can be delayed. As a rule, heat erythema that usually develops rapidly, is delayed so long that a particular region can be treated with the laser without interruption. Erythema often means that small vessels cannot be treated because they “disappear” in the erythema. Thus, the delaying of erythema is particularly important when treating blood vessels.

According to an embodiment of the present invention, the film should also satisfy hygienic requirements. The film can be a disposable article and may be replaced several times during a single treatment to ensure ideal cooling, transparency and contact are always optimum.

The applicants performed significant testing that shows successful results. A transmission test using a transparent wound cover Geliperm®, polyacrylamide agar moist gel Plate 100×30×3.3 mm in size, test beam profiles of 1 Hz 44/Jcm², 1 Hz 35/Jcm² and 1 Hz 10/Jcm² were used with no discernible differences in the beam profile compared with final images without Geliperm®.

Test Transmission Results:

| without | 10.5 J | with | 10.2 J | |
|---|---|---|---|---|
| Geliperm ® | 10.3 J | Geliperm ® | 10.4 J | |
| | 10.5 J | | 10.2 J | |
| | 10.4 J | | 10.3 J | Transmission: 99% |
| | 43.5 J | | 43.5 J | |
| | 43.6 J | | 43.5 J | |
| | 43.6 J | | 43 J | |
| | 43.6 | | 43.3 J | Transmission: 99% |

Test for Heating on Uninterrupted Operation:

Geliperm® was placed on a taper and fired at 4 Hz 15 Jcm² for 20 minutes. After 20 minutes no discernable changes to Geliperm, no drying out, and no heating of the Geliperm®.

Test for Burning Hair:

Tufts of hair were placed on a polytetrafluroethylene (PTFE) disc and covered with Geliperm®. A 12 mm hand piece was put on and the laser fired at 1 Hz 44 J/cm². After 10 shots, Geliperm® had burned half-way through at the same site there was no damage to the taper.

Test for Heat Capacity (without Skin Contact):

Measurements were taken with a thermo-element and IR thermometer at room temperature 22° C. The results are shown in the table below:

| t [min] | T Gelip. [° C.] | Comment |
|---|---|---|
| 0 | −3.5 | opaque and brittle |
| 2.5 | −3 | |
| 5 | −2.5 | |
| 10 | −2 | |
| 15 | −1 | |
| 20 | 0 | clear and soft |
| 25 | 2 | |
| 30 | 7.5 | |
| 35 | 12 | |
| 40 | 14 | |
| 45 | 15.5 | |
| 50 | 16 | |
| 55 | 16.5 | |
| 60 | 17 | |

Test for heat capacity (with skin contact):

measurements with ir thermometer were taken at room temperature 22° C. The results are shown in the table below:

| t [sec] | T Gelip. [° C.] | T skin [° C.] |
|---|---|---|
| 0 | 10 | 33 |
| 10 | 14 | |
| 20 | 17 | |
| 30 | 17 | |
| 40 | 18 | |
| 50 | 19.5 | |
| 60 | 20.5 | |
| 70 | 20.5 | |
| 80 | 21.5 | |
| 90 | 21.5 | |
| 100 | 22.5 | |
| 110 | 22.5 | |
| 120 | 23 | |
| 130 | 23 | 27 |

Test for Skin Temperature:

Measurements were taken with an IR thermometer, and results are shown in the table below:

| t [min] | T skin. [° C.] | T. Gelip. [° C.] |
|---|---|---|
| 0 | 33 | 9 |
| 5 | 26 | |
| 10 | 25 | |
| 15 | 27 | |
| 20 | 26 | |
| 25 | 26 | 17 |

One skilled in the art will readily understand that the technique of the present invention considerably improves and simplifies the use of laser that work with either a contact gel or a cooling system, or for which cooling or better transparency is desirable, or the occasional production of the smell caused by heating structures containing keratin is to be avoided. Polyacrylamide agar moist gel moulds itself ideally to all surfaces, and causes no unpleasantness such as gel running off, thinning of the transmission layer with loss of the effect, or increasing pain. Treatment takes less time, is considerably more pleasant than with liquid gels for doctor and patient alike, is perfectly hygienic, requires no cleaning, and saves and protects the sensitive and expensive laser contact 16 surfaces. During therapy, there is no attenuation to the laser beam as it passes through the clear film and hits the skin and structures being treated.

Geliperm® has been approved for medical application to the skin and wounds, and is available as a disposable article. All sites, extensive and small areas, can be treated automatically and quickly. The present invention is undoubtedly user-friendly and patient compliance. In tests, patients' evaluated the two techniques—liquid gel or the new product—and unequivocally chose the film over the liquid gel.

Thus, the present invention has been fully described with reference to the drawing. Although the invention has been described based upon these preferred embodiments, it would be apparent to those of skilled in the art that certain modifications, variations, and alternative constructions would be apparent, while remaining within the spirit and scope of the invention. In order to determine the metes and bounds of the invention, therefore, reference should be made to the appended claims.

1. In combination:
a laser device having a contact surface, for performing laser treatment of an area of a surface of a patient; and
a protecting/cooling article of a substantially transparent gel film sheet which is cut to an appropriate size for covering said area of said surface of said patient, which gel is not a liquid gel material; the gel film sheet for positioning between the laser device contact surface and said surface of said patient to be treated; wherein the gel film sheet is for applying to said area of said surface of said patient to be treated prior to performing laser treatment to said area; wherein laser treatment is performed to said area by passing a laser beam from said laser device to said surface of said patient through said gel film sheet without substantial attenuation of said laser beam when transmitted through said gel film sheet; wherein said gel film sheet, when applied to said surface of said patient to be treated, provides cooling of said surface of said patient during laser treatment; and wherein said gel film sheet protects said contact surface of said laser device during said laser treatment.

2. The combination of claim 1, wherein said laser device is a photothermolysis laser (PTL) device.

3. The combination of claim 1, wherein said transparent gel film sheet is a hydrophilic, organic transparent gel.

4. The combination of claim 1, wherein said substantially transparent gel film sheet is a polyacrylamide agar moist gel.

5. The combination of claim 1, wherein said gel film sheet has a thickness in a range of approximately 0.1-25 mm.

6. The combination of claim 1, wherein said substantially transparent gel film sheet has a thickness in a range of approximately 0.5-10 mm.

7. The combination of claim 1, wherein said substantially transparent gel film sheet has a thickness in a range of approximately 1-5 mm.

8. The combination of claim 5, wherein said substantially transparent gel film is a polyacrylamide agar moist gel.

9. The combination of claim 6, wherein said substantially transparent gel film is a polyacrylamide agar moist gel.

10. The combination of claim 1, wherein said protecting/cooling article consists essentially of said substantially transparent gel film sheet.

11. In combination:
a laser device having a contact surface, for performing laser treatment of an area of a skin surface of a patient; and
a protecting/cooling article of a substantially transparent gel film sheet which is cut to an appropriate size for covering said area of said skin surface of said patient, which gel is not a liquid gel material; the gel film sheet for positioning between the laser device contact surface and said skin surface of said patient to be treated; wherein the gel film sheet is for applying to said area of said skin surface of said patient to be treated prior to performing laser treatment to said area; wherein laser treatment is performed to said area by passing a laser beam from said laser device to said skin surface of said patient through said gel film sheet without substantial attenuation of said laser beam when transmitted through said gel film sheet; wherein said gel film sheet, when applied to said skin surface of said patient to be treated, provides cooling of said skin surface of said patient during laser treatment; and wherein said gel film sheet protects said contact surface of said laser device during said laser treatment.

12. The combination of claim 11, wherein said laser device is a photothermolysis laser (PTL) device.

13. The combination of claim 11, wherein said transparent gel film sheet is a hydrophilic, organic transparent gel.

14. The combination of claim 11, wherein said substantially transparent gel film sheet is a polyacrylamide agar moist gel.

15. The combination of claim 11, wherein said gel film sheet has a thickness in a range of approximately 0.1-25 mm.

16. The combination of claim 11, wherein said substantially transparent gel film sheet has a thickness in a range of approximately 0.5-10 mm.

17. The combination of claim 11, wherein said substantially transparent gel film sheet has a thickness in a range of approximately 1-5 mm.

18. The combination of claim 15, wherein said substantially transparent gel film is a polyacrylamide agar moist gel.

19. The combination of claim 16, wherein said substantially transparent gel film is a polyacrylamide agar moist gel.

20. The combination of claim 11, wherein said protecting/cooling article consists essentially of said substantially transparent gel film sheet.
